(19) United States
(12) Patent Application Publication
(10) Pub. No.: US 2009/0299294 A1
(43) Pub. Date: Dec. 3, 2009

(54) SUPPORT DEVICES FOR CATHETERS

(75) Inventor: Lesley Ruth Pinkus, Stevenage (GB)

Correspondence Address: STETINA BRUNDA GARRED & BRUCKER, 75 ENTERPRISE, SUITE 250, ALISO VIEJO, CA, 92656, US

(73) Assignee: East & North Hertfordshire NHS Trust, Stevenage (GB)

(21) Appl. No.: 12/225,648

(22) PCT Filed: Mar. 28, 2007
(86) PCT No.: PCT/GB2007/001128

§ 371 Date: Jan. 21, 2009

(30) Foreign Application Priority Data
Mar. 28, 2006 (GB) 0606131.1

(51) Int. Cl. A61M 25/02

(52) U.S. Cl. 604/177

(57) ABSTRACT

A device for securing a catheter tube against a patient's body, said device comprising a base member for attaching to the patient's body in use, and a gripping member, the gripping member comprising: a first region that is attached to, or attachable, to, or integral with, the base member; an attachment region that is attachable to the base member; and an intermediate region between the first region and the attachment region, the intermediate region being adapted to wrap over a catheter tube; the device being arranged such that, in use, the first region of the gripping member is attached to the base member, the intermediate region is wrapped over a catheter tube, and the attachment region is attached to the base member, thereby securing the catheter tube between the base member and the gripping member. Also provided is a device for securing two catheter tubes against a patient's body, said device comprising a base member for attaching to the patient's body in use, and two gripping members, each gripping member comprising: a first region that is attached to, or attachable to, or integral with, the base member; an attachment region that is attachable to the base member; and an intermediate region between the first region and the attachment region, the intermediate region being adapted to wrap over a catheter tube; the device being arranged such that, in use, the first region of each gripping member is attached to the base member, the intermediate region of each gripping member is wrapped over a respective catheter tube, and the attachment region of each gripping member is attached to the base member, thereby securing the two catheter tubes between the base member and the respective gripping members. This device is particularly suited for use with single or dual-lumen catheters, for example as used in haemodialysis.

### BACKGROUND OF THE INVENTION

This invention relates to devices for securing catheters against patients' bodies. The invention is particularly suitable, but by no means limited, for use with tunnelled central venous catheters such as those used for haemodialysis.

A catheter is a hollow flexible tube, usually made of plastic, for insertion into a body cavity, duct, or vessel. Catheters may be used for a variety of medical purposes, examples of which include insertion into a lumen of a blood vessel for performing haemodialysis or to administer an injection, drainage of urine from the bladder through the urethra, or insertion through a blood vessel into the heart for diagnostic purposes.

For haemodialysis, a pair of tunnelled central venous catheters (CVCS) such as the Tesio catheter system, or a dual-lumen catheter (e.g. HemoSplit™ or Ash Split), may be used. Each catheter tube (or “line”) is commonly inserted into a lumen of a vein (e.g. internal jugular vein, subclavian vein or femoral vein). To prevent infection getting into the bloodstream each catheter is usually “tunnelled” under the skin, to emerge further down on the chest. A “cuff” is often provided around each catheter tube to anchor it in position just under the skin surface, and in order to help prevent infection tracking along the line. When not in use, the end of each catheter tube outside the body is closed with a clamp and Luer Lock, in order to prevent air, bacteria or other undesirable matter from entering, and to prevent blood escaping.

For use in dialysis, the end of each catheter tube outside the body is unclamped and coupled to a dialysis machine. During dialysis, one catheter tube carries blood from the patient to the dialysis machine, and the other catheter tube returns the blood from the dialysis machine to the patient.

In use, catheters are liable to be pulled accidentally, which can result in an injury or even a medical emergency for the patient, a potentially critical loosening of the cuffs around the catheter tube, or an increased likelihood of infection around the catheter site. As an indication of the number of incidents of infection, in the United States alone about 250,000 CVC-related bloodstream infections are reported per year, at an estimated cost to the US healthcare system of US $25,000 per incident. This is a significant number, and represents an appreciable proportion of the 5 million or so CVCs that are purchased every year in the US. (Reference: Slaughter S. E., Intravascular catheter-related infections: strategies for combating this common foe, Postgrad Med 2004;116(5):59-66.)

Conventionally, in order to reduce the likelihood of a catheter being pulled accidentally, and to mitigate the above consequences should the catheter be pulled accidentally, catheters have been secured directly onto the patient's skin using standard medical adhesive tape. However, such tape may normally only be used once, and does not readily allow the catheter to be removed or repositioned without cutting or removing the tape. Removal of the tape can be uncomfortable for the patient, particularly if the adhesive on the tape has become stuck to the patent's body hair. Furthermore, adhesive from the tape can remain stuck on the outside of the catheter, causing the catheter to become sticky and unsightly, and ultimately harder to keep clean and maintain the standard of hygiene required in a medical environment.

In a previous attempt to overcome some of these problems, a hard rigid plastic disc was developed that incorporates catheter retaining clips into which a catheter line may be pressed and thereby clipped in place. The underside of the rigid plastic disc is coated with adhesive and stuck to the patient's body. Such discs have proved to be uncomfortable for patients, particularly at the instance when a catheter line is pressed onto a disc. These discs are generally only suitable for single use, since the catheter line cannot be readily removed. Additionally, because the retaining clips are of a fixed shape and configuration, and because catheters tubes are available in a range of external diameters, it has been found that the discs are unable to hold some catheter lines sufficiently securely, and that the lines are still prone to slipping. It is also possible to damage some catheter lines when pressing them into the retaining clips.

A further problem may be encountered by a medical professional when installing a pair of catheter lines for haemodialysis. The two catheter lines are normally attached concurrently to the patient, but manipulating the two lines simultaneously can be cumbersome and awkward. Additionally, if the catheter lines are placed in contact with one another and attached to the patient's body by tape enveloping both the lines, the lines (being plastic) are prone to slip against each other beneath the tape, and this can lead to the lines becoming disconnected from the patient. Indeed, it is estimated that approximately 10% of catheter lines fall out during or between haemodialysis procedures. This is not only dangerous for the patient, due to the accidental loss of blood, but also exposes the patient to an increased risk of infection.

### SUMMARY OF THE INVENTION

According to a first aspect of the present invention there is provided a device for securing a catheter tube against a patient's body, said device comprising a base member for attaching to the patient's body in use, and a gripping member, the gripping member comprising: a first region that is attached to, or attachable to, or integral with, the base member; an attachment region that is attachable to the base member; and an intermediate region between the first region and the attachment region, the intermediate region being adapted to wrap over a catheter tube; the device being arranged such that, in use, the first region of the gripping member is attached to the base member, the intermediate region is wrapped over a catheter tube, and the attachment region is attached to the base member, thereby securing the catheter tube between the base member and the gripping member.

The term “gripping” as used herein should be interpreted broadly, to encompass any kind of action that restrains or impedes the tendency of a catheter tube to slip, slide or otherwise become dislodged from the patient. The act of gripping may be achieved simply using friction between the catheter and the device. However, additional gripping aids, such as foam or rubberised padding, or adhesive, may be incorporated on the underside of the gripping member, contacting the catheter in use, although such additional gripping aids are not considered essential to the invention.

By virtue of the gripping member being able to be wrapped over the catheter tube and then attached to the base member, catheters of a range of external diameters can easily be securely attached to the device and thus to the patient, without causing any damage to the catheter line. Additionally, the inherent adjustability of the gripping member when wrapping it over the catheter line enables the line to be secured to the patient at an optimum degree of secureness, and this is not only safer but also more reassuring for the patient. Through being able to be made from flexible or pliable materials, the device is more comfortable to fit to a patient than a hard plastic disc. Further, since the region of the gripping member which contacts the catheter may be free of adhesive, this results in less (or no) adhesive residue remaining on the catheter line when removed, and gives a neater, tidier and less unsightly dressing than has been the case with traditional dressings.

According to a second aspect of the present invention there is provided a device for securing two catheter tubes against a patient's body, said device comprising a base member for attaching to the patient's body in use, and two gripping members, each gripping member comprising: a first region that is attached to, or attachable to, or integral with, the base member; an attachment region that is attachable to the base member; and an intermediate region between the first region and the attachment region, the intermediate region being adapted to wrap over a catheter tube; the device being arranged such that, in use, the first region of each gripping member is attached to the base member, the intermediate region of each gripping member is wrapped over a respective catheter tube, and the attachment region of each gripping member is attached to the base member, thereby securing the two catheter tubes between the base member and the respective gripping members.

In addition to the advantages outlined above in relation to the first aspect of the invention, this second aspect of the invention greatly facilitates the attachment of a dual-lumen catheter to a patient, since each catheter line can be fastened separately to the base member and thus to the patient. Moreover, since each catheter line is fastened by its own gripping member, the lines are not mounted in sliding contact with one another, and are consequently less prone to slipping.

With the second aspect of the invention, preferably the gripping members are flexibly attached in substantially the centre of the base member. By virtue of the gripping members extending in opposite directions away from the centre of the base member, this enables two catheter tubes to be easily secured in parallel to the device.

With either the first or the second aspect of the invention, the attachment region or regions may be substantially flat. This may facilitate adhesion of the gripping member(s) to the base member.

Preferably the attachment region or regions is/are coated with adhesive. Alternatively, or in addition, the base member may be coated with adhesive in a region or regions to correspond with the attachment region or regions.

Preferably the adhesive is adapted to allow the gripping member or members to be attached to and then detached from the base member. This highly advantageously enables the catheter tube(s) to be easily removed from the patient's body.

Moreover, the adhesive may be adapted to allow the gripping member or members to be attached to, detached from, and then re-attached to the base member. Such an adhesive not only enables the catheter tube(s) to be easily removed from the patient's body, but also readily enables the tubes to be adjusted or repositioned on the patient, thereby enabling a medical professional to fit the tubes in an optimum manner for operation and patient comfort.

As an alternative to using such adhesive, the attachment region or regions may incorporate hook-and-loop fastener material (e.g. Velcro (RTM)), and the base member may incorporate complementary hook-and-loop fastener material. This also enables the catheter tube(s) to be easily removed, adjusted or repositioned on the patient.

Preferably, but not necessarily, the or each intermediate region incorporates a preformed curvature to substantially correspond with the outside circumference of a catheter tube. This advantageously aids the location and gripping of the catheter.

The or each intermediate region may also incorporate gripping means on its underside, for enhancing the grip between the gripping member and a catheter tube in use. The gripping means may be selected from a group comprising: padding, foam, rubber, adhesive. This list is not intended to be exhaustive, and those skilled in the art may identify other gripping means that are suitable for this purpose. Indeed, this disclosure is intended to apply to all existing gripping means that are suitable for this purpose, and also those that have yet to be invented or developed. Such gripping means may additionally, or alternatively, be provided on the upper surface of the base member, positioned so as to contact the catheter tube in use and thereby further enhance the grip between the device and the catheter.

The or each gripping member may extend outwardly beyond the base member, thereby providing tab or handle means to facilitate pulling open the device and releasing the catheter.

The device may further comprise a cover part for covering the site(s) at which one or more catheter tubes emerge from the human body. This advantageously helps to prevent the human body from becoming infected at the catheter exit site(s).

Preferably the device is impregnated or treated with an anti-infection or antibacterial agent, to reduce the likelihood of infection to the patient.

The device may also be impregnated or treated with a waterproofing agent, to protect it from becoming dysfunctional should it become wet.

The device may be made of a material selected from a group comprising: gauze, paper, reinforced paper, cardboard, woven fabric, non-woven fabric, plastic, terry cloth. Again, this list is not intended to be exhaustive, and those skilled in the art may identify other materials that are suitable for this purpose. This disclosure is intended to apply to all existing materials that are suitable for manufacturing this device, and also materials that have yet to be invented or developed.

The material used to make the device as a whole, or alternatively just the gripping member(s), may also be provided with a degree of elasticity, to enhance the grip between the device and the catheter(s).

Preferably the underside of the base member incorporates adhesive for attaching the device to the patient's body in use. This facilitates the application of the device to the patient.

According to a third aspect of the invention there is provided a device configured to secure a catheter tube against a patient's body and also cover the site at which the catheter tube emerges from the patient's body.

According to a fourth aspect of the invention there is provided a method of securing a catheter tube against a patient's body, said method comprising: obtaining a device in accordance with the first, second or third aspects of the invention; attaching a first region of the gripping member to the base member or receiving the device with the first region of a gripping member pre-attached to the base member; attaching the base member to the patient; wrapping the gripping member over the catheter tube; and attaching the attachment region of the gripping member to the base member, thereby securing the catheter tube between the base member and the gripping member.

### BRIEF DESCRIPTION OF THE DRAWINGS

Embodiments of the invention will now be described, by way of example only, and with reference to the drawings in which:

FIG. 1 illustrates a first embodiment of a support dressing, as initially supplied, for securing a single catheter tube to a patient;

FIG. 2 illustrates the support dressing of FIG. 1 fastened around a catheter tube as in use;

FIG. 3 illustrates an end-on cross-sectional view of the device shown in FIG. 2;

FIG. 4 illustrates an end-on cross sectional view of a variant of the device of FIGS. 1, 2 and 3, in which, in this case, the gripping member is initially provided separate from the base member and is configured to be attached to the base member on both sides of the catheter tube;

FIG. 5 illustrates a second embodiment of a support dressing, as initially supplied, for securing two catheter tubes to a patient;

FIG. 6 shows the support dressing of FIG. 5 fastened around two catheter tubes as in use;

FIG. 7 shows an end-on cross sectional view of the device shown in FIG. 6;

FIG. 8 illustrates an end-on cross sectional view of a variant of the device of FIGS. 5, 6 and 7, in which, in this case, the gripping members are initially provided separate from the base member, and are configured to be attached to the base member on both sides of each catheter tube;

FIG. 9 illustrates a plan view of another variant of the device of FIGS. 5, 6 and 7, adapted to hold the two catheter tubes further apart; and

FIG. 10 illustrates a plan view of a variant of the device of FIG. 9, this variant having a cover part suitable for covering the site at which the catheter tubes emerge from the body.

FIGS. 11a and 11b illustrate a further variant of the device of FIGS. 5, 6 and 7;

FIGS. 12 and 13 illustrate a third embodiment of a support dressing as initially supplied; and

FIG. 14 illustrates a fourth embodiment of a support dressing, as initially supplied, including means for protecting the end of a catheter.

In the figures, like elements are indicated by like reference numerals throughout.

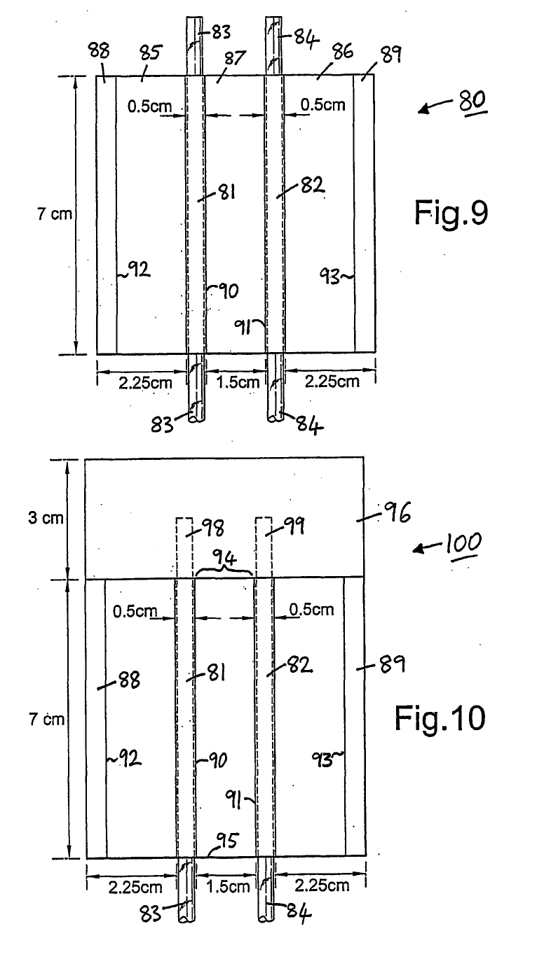

The dimensions in FIGS. 9 and 10 are provided by way of example only, as the devices may be made in a variety of shapes and sizes.

### DETAILED DESCRIPTION OF PREFERRED EMBODIMENTS

The present embodiments represent the best ways known to the applicant of putting the invention into practice. However they are not the only ways in which this can be achieved.

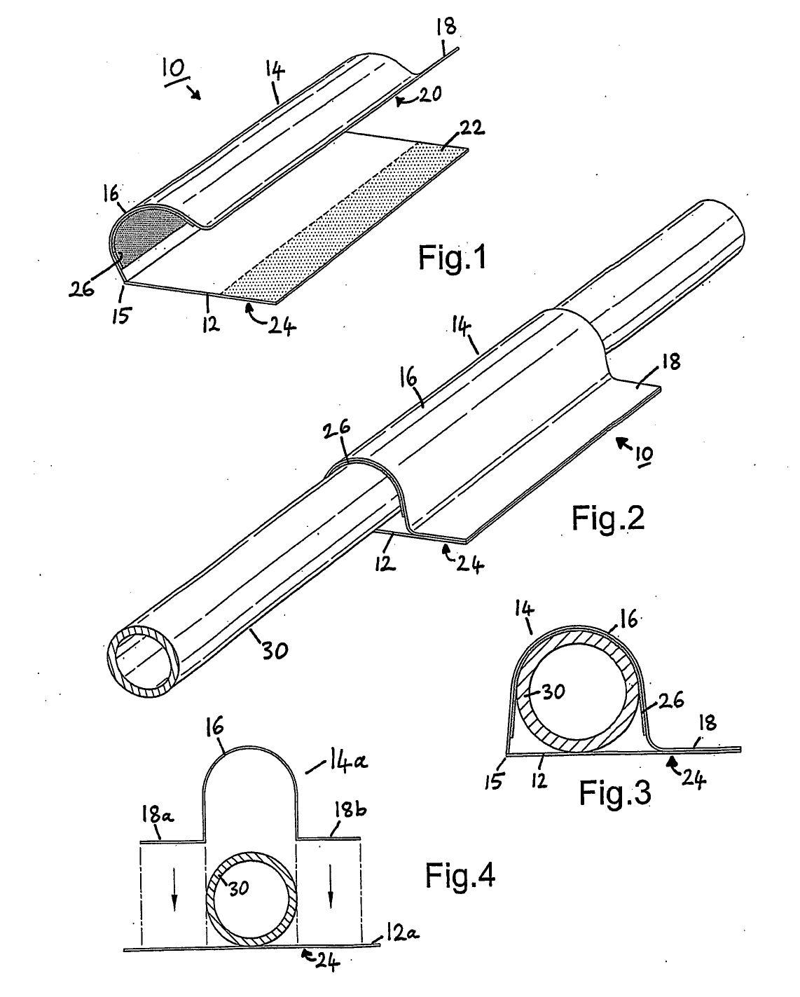

FIG. 1 illustrates a first embodiment of a support dressing or support device 10 for use with a single catheter tube. The device 10 comprises a base member 12 and a gripping member 14. In this illustrated embodiment, the gripping member 14 is pre-attached by a flexible fold, hinge or seam to the base member 12, such that the gripping member 14 may be closed onto the base member 12 in use.

The base member 12 is formed of a generally flat piece of material, which is preferably flexible, that is attachable to a patient's body in use.

The gripping member 14 is of the general form of a strap or cover, which is preferably flexible. The gripping member 14 comprises an attachment region 18, of which the underside 20 is attachable to the base member 12—for example attaching to region 22 of the base member. Between the end 15 and the attachment region 18, the gripping member 14 incorporates an intermediate region 16 that is adapted to wrap over a catheter in use.

As illustrated in FIG. 1, the intermediate region 16 may be provided with a preformed curvature to substantially correspond with the outside circumference of a catheter tube, thereby aiding the location and gripping of the catheter tube. A range of devices 10 may be supplied having different preformed curvatures, for use with different diameters of catheters. However, it should be emphasised that having a preformed curvature is not essential, and indeed the gripping member 14 may simply consist of a substantially flat flexible strap made of any suitable material.

Additionally, or alternatively, the underside of the intermediate region 16 may be provided with additional gripping means 26, to further enhance the grip between the device 10 and a catheter tube in use. This additional gripping means 26 may be, for example, padding material, foam, rubber, or a tacky or adhesive coating. However, in the currently preferred embodiments the underside of the intermediate region 16 is substantially free of adhesive, thereby enabling a catheter tube to be gripped without leaving any sticky residue on the catheter when removed.

As illustrated in FIGS. 2 and 3, the support dressing 10 may be used to secure a catheter tube 30 against a patient's body. In use, the underside 24 of the base member 12 is generally first attached to the patient's skin, using adhesive that may be pre-coated on the underside 24, or some other adhesive or fastening means that may be applied by a medical professional as appropriate. A catheter tube 30 is then introduced between the base member 12 and the gripping member 14. The gripping member 14 is then wrapped securely over the catheter tube 30, and then the underside 20 of the attachment region 18 is attached to the corresponding region 22 of the base member 12. The gripping member 14 is intended to be wrapped over the catheter tube 30 sufficiently tightly and snugly to provide sufficient grip between the catheter tube 30 and the device 10 to prevent the catheter line from slipping, but without causing damage to the catheter. The material used to make the gripping member 14, or the device 10 as a whole, may be provided with a suitable degree of elasticity, to further enhance the grip between the catheter tube and the device 10.

The length of the device 10, in the direction parallel to the catheter tube 30 in use, may be longer than illustrated, to provide more adhesion with the patient's body. The width of the device 10, in the direction perpendicular to the catheter tube 30 in use, may also be wider than illustrated, again to provide more adhesion to the patient's body.

The regions 16, 18 of the gripping member 14 need not be formed or marked as distinct regions, and indeed the gripping member may have a uniform appearance along its length. In a similar manner, the region 22 of the base member 12 need not be formed or marked as distinct from any other region of the base member.

As illustrated in FIG. 4, the gripping member need not be supplied with one end pre-attached to the base member 12a. Instead, a gripping member 14a may be supplied separate from the base member 12, and each side of the gripping member 14a may incorporate separate attachment regions 18a, 18b for attaching to the base member 12 either side of the catheter tube in use. Such an arrangement advantageously enables the gripping member 14a to be removed and replaced as necessary, without necessarily having to remove and replace the base member 12. It also enables a range of gripping members 14a incorporating different preformed curvatures to be interchangeably used with a single geometry of base member.

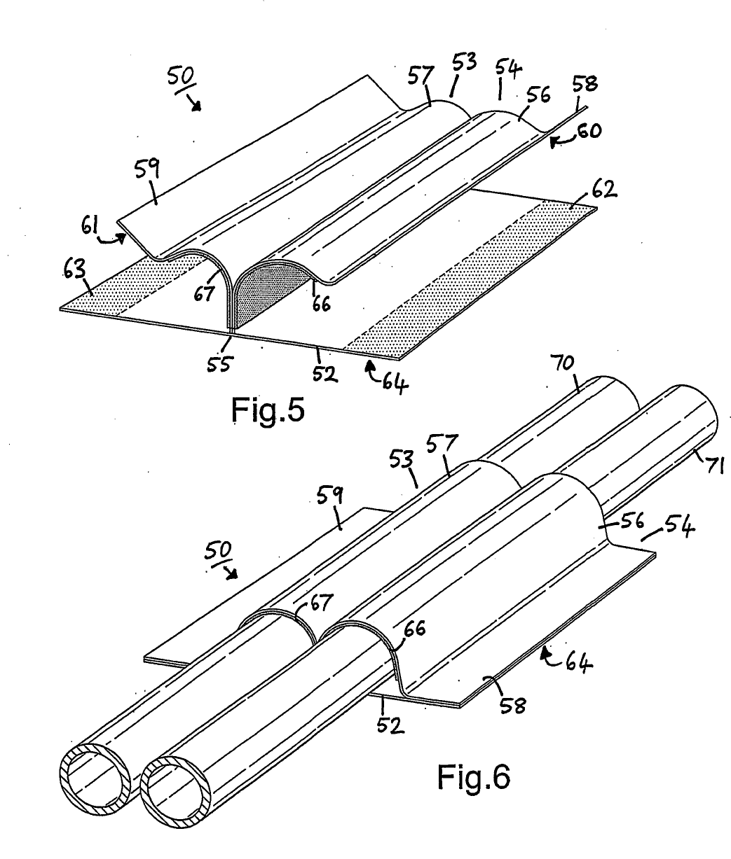

FIG. 5 illustrates a second embodiment of a support dressing or support device 50, for use with a pair of catheter tubes. Here, the device 50 comprises a base member 52 and a pair of gripping members 53, 54. The gripping members 53, 54 are preferably made from a single piece of material, and are pre-attached to the base member 52 via a flexible hinge or seam region 55. The seam or hinge 55 may be formed by a line of stitching or adhesive, and is aligned parallel to the orientation of the catheters in use. The device 50 is substantially symmetrical about the seam or hinge 55.

The general features and principle of operation of the second embodiment are substantially the same as those of the first embodiment.

As with the first embodiment, the gripping members 53, 54 are preferably of the general form of flexible straps or covers. Each gripping member 53, 54 comprises a respective attachment region 59, 58, of which the undersides 61, 60 are attachable to the base member 52—for example attaching to regions 63 and 62 of the base member. Between the hinge end 55 and the attachment regions 58, 59, each gripping member 54, 53 incorporates an intermediate region 56, 57 that is adapted to wrap over a respective catheter in use.

The intermediate regions 56, 57 may each be provided with a preformed curvature to substantially correspond with the outside circumference of a catheter tube, thereby aiding the location and gripping of the catheter tubes. As described above in relation to the first embodiment, the underside of the intermediate regions 56, 57 may also, or alternatively, be provided with additional gripping means 66, 67 to enhance grip. In the currently preferred embodiment, the undersides of the intermediate regions 56, 57 are substantially free of adhesive, thereby enabling the catheter tubes to be gripped without leaving any sticky residue on the catheters when removed.

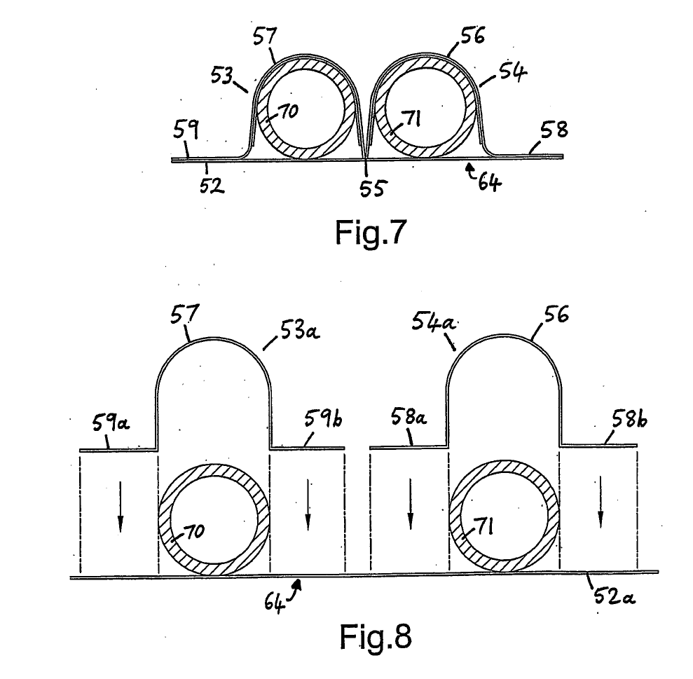

As illustrated in FIGS. 6 and 7, the support dressing 50 can be used to secure a pair of catheter tubes 70, 71, for example for use in haemodialysis. The underside 54 of the base member 52 is first attached to the patient's body. A catheter tube (e.g. 70) is then introduced between the base member 52 and one of the gripping members (e.g. 53). The gripping member 53 is then wrapped securely over the catheter tube 70, and then the underside 61 of the attachment region 59 is attached to the corresponding region 63 of the base member 52.

As before, the gripping members 53, 54 are intended to be wrapped over the catheter tubes 70, 71 sufficiently tightly and snugly to provide sufficient grip between the catheter tubes and the device 50 to prevent the catheter lines from slipping, but without causing damage to the catheters. The gripping members 53, 54 may be provided with a degree of elasticity to further enhance the grip between the catheter tubes and the device 50.

By virtue of the hinge region 55 being between the catheter tubes 70, 71, the tubes are held slightly apart from one another, and are kept out of contact with each other within the device 50. Accordingly, the tubes (which have a smooth plastic exterior) are prevented from sliding against one another. Thus, using the present device 50 results in a much more secure fastening of the catheter tubes than would be the case were the tubes wrapped in sliding contact with each other, using a single dressing spanning or enveloping both tubes.

As illustrated in FIG. 8, gripping members 53a, 54a may be supplied separate from the base member 52a, and each side of the gripping members (e.g. 53a) may incorporate separate attachment regions (e.g. 59a, 59b) for attaching to the base member 52a either side of a catheter tube (e.g. 70) in use. This advantageously enables the gripping members 53a, 54a to be removed and replaced as necessary, without necessarily having to remove and replace the base member 52a. It also enables a range of gripping members incorporating different preformed curvatures to be interchangeably used with a single geometry of base member 52a.

FIG. 9 illustrates a plan view of a variant of the device of FIGS. 5, 6 and 7. This device 80 is similar to the device 50 shown in FIGS. 5, 6 and 7, but is adapted to hold the two catheter tubes further apart than is the case with device 50, since catheter tubes are often not inserted in such close proximity as those illustrated in FIGS. 6 and 7. As with device 50, this device 80 has a base member (underneath and therefore not shown, but extending from line 92 across to line 93) and a pair of gripping members 81-88 and 82-89.

Gripping member 81-88 is attached to the base member using a hinge or seam at line 90, or to the right of line 90. Similarly, gripping member 82-89 is attached to the base member using a hinge or seam at line 91, or to the left of line 91. Thus, the hinges or seams are within, or at opposing sides of, the central region 87 of the device 80.

Gripping member 81-88 comprises an attachment region 85 by which it may be attached to the base member. Between the attachment region 85 and the hinge or seam (which is at or near line 90) is an intermediate region 81 for wrapping over a catheter 83 in use. The gripping member further comprises an edge region 88, beyond the attachment region 85, at the far end of the gripping member 81-88 from the catheter 83. This edge region 88 extends outwards beyond the edge of the base member (which is at line 92), thereby providing a tab with which a medical professional can pull open the device to release the catheter tube 83.

Similarly, gripping member 82-89 comprises an attachment region 86 by which it may be attached to the base member. Between the attachment region 86 and the hinge or seam (which is at or near line 91) is an intermediate region 82 for wrapping over a catheter 84 in use. The gripping member further comprises an edge region 89, beyond the attachment region 86, at the far end of the gripping member 82-89 from the catheter 84. This edge region 89 extends outwards beyond the edge of the base member (which is at line 93) thereby providing a tab with which the device can be pulled open in order to release the catheter tube 84.

The edge/tab regions 88, 89 may each be about 0.5 cm in width, although this measurement is merely as an example and other widths may be used instead. Thus, with reference to FIG. 9, the base member may be about 7 cm in the direction parallel to the catheter tubes, by about 6 cm wide (i.e. from line 92 across to line 93).

It will be appreciated that the spacing of the catheters 83, 84 (which, in the example shown in FIG. 9, is 1.5 cm) may be determined by the width of the central region 87 and the separation of the two hinges or seams.

As described above in relation to the previous embodiments, the intermediate regions 81, 82 may be provided with a preformed curvature to substantially correspond with the outside circumference of the catheter tubes. However, as with the previous embodiments, a preformed curvature is not considered essential. The underside of the intermediate regions 81, 82 may also, or alternatively, be provided with additional gripping means to enhance grip.

FIG. 10 illustrates a plan view of a variant 100 of device 90 of FIG. 9. In this variant 100, the base member (not shown) extends from line 92 across to line 93, and from line 94 down to line 95. The gripping members 81-88 and 82-89 are as described above in relation to FIG. 9. However, device 100 of FIG. 10 also incorporates a cover part 96 which is shaped and configured to provide having a cover for the, sites 98, 99 at which the catheter tubes 83, 84 emerge from the human body. The cover part 96 may be integrally attached to the rest of the dressing, for example along line 94, and may be provided with an adhesive underside (or just adhesive around the edge of the underside), to enable it to be secured over the catheter exit sites 98, 99. Alternatively, instead of having an adhesive underside, the cover part 96 may be secured to the body using conventional medical adhesive tape.

Instead of just being attached along line 94, the cover part 96 may be attached along substantially the entire side of the base member that is closest to the catheter exit sites in use, and may incorporate gaps, spaces or holes through which the catheter tubes may pass as necessary.

Similar cover parts, shaped and configured to cover the catheter exit site(s) in use, may also be incorporated in the other embodiments described previously—including those embodiments illustrated in FIGS. 1 to 4 for securing only a single catheter tube, and the embodiments illustrated in FIGS. 5 to 8 for securing a pair of catheter tubes.

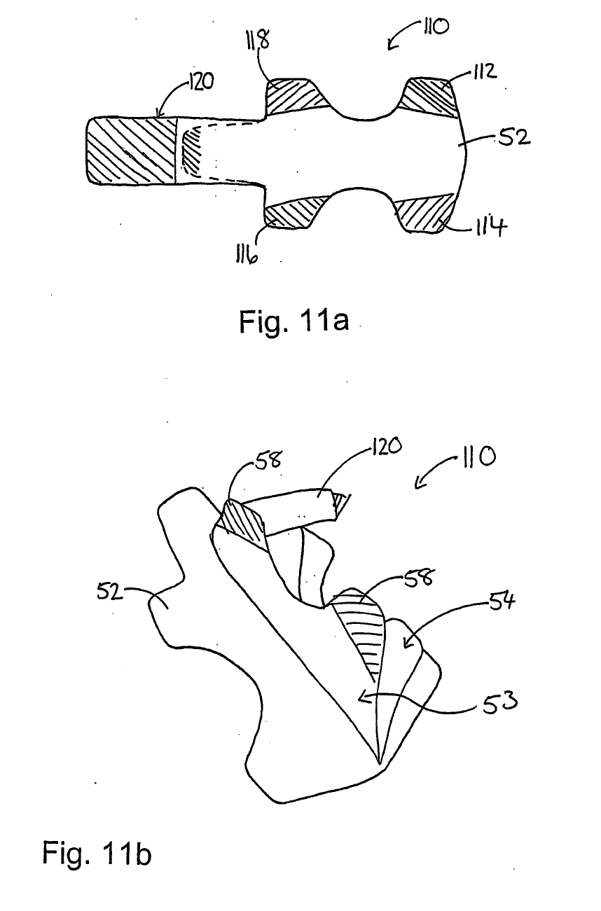

FIGS. 11a and 11b illustrate a further variant of FIGS. 5, 6 and 7. This device 110 is similar to the device shown in FIGS. 5, 6 and 7 but is shaped to conform to the contours of the body to which it is attached. As can be seen, the base member 52 is provided with concave sides. This is particularly useful for use with catheters which exit the body close to a female's breasts.

The device is further provided with a pair of gripping members 53,54. The gripping members 53, 54 are substantially as described with reference to FIGS. 5,6 and 7. The gripping members 53, 54 are attached to the base member 52 using a hinge or seam within the central region of the base member 52 such that each gripping member 53, 54 can extend over a catheter placed on one side of the central region of the base member 52.

Each gripping member 53, 54 includes at least attachment region 58 by which it may be attached to the base member. Between the attachment region 58 is an intermediate region 56 for wrapping over a catheter in use, as described with reference to FIGS. 5, 6 and 7. if desired the gripping member 53, 54 may be provided with an edge region as described with reference to FIG. 8.

The base member 52 may be provided with regions 112, 114, 116, 118 arranged to attach the base member 52 to the patient's body. The attachment regions 112, 114, 116, 118 are provided in a configuration which enables secure attachment of the base member 52 to another object whilst enabling the minimum amount of adhesive to be used. The reduction in the amount of adhesive contacting a patient's skin is advantageous as it reduces the amount of pain felt by the patient when the base member 52 is removed. Similar adhesive regions may also be incorporated in the other embodiments illustrated in FIGS. 1 to 10 described previously.

Advantageously, the device may be provided with a cover part 120. The cover part 120 may be, for example, a tab which extends from the edge of the base member 52 closest to the catheter exit site. The cover part 120 may be integral with the base member 52 or attached to the base member 52, for example by stitching or adhesive. Alternatively, the cover part 120 may be integral or attached to one or more of the gripping members 53 as illustrated in FIG. 11b.

The cover part 120 extends over the catheter exit site and covers the exit site. The catheter then passes underneath the cover part 120 and passes past the end of the cover part 120 extending over the base member. Alternatively, the cover part 120 may include at least one gap, space, or hole 122 through which the catheter tube may pass as necessary.

The cover part 120 is attached to the patent's body using adhesives. The adhesive may cover the entirety of the underside of the cover part 120 as illustrated in FIG. 11a. Alternatively adhesive regions, such as those described with reference to the base member, may be used to attach the cover part to a patient's body.

Similar cover parts, shaped and configured to cover the catheter exit site in use, may also be incorporated in the other embodiments described previously.

FIGS. 12 & 13 illustrates a third embodiment of a support dressing or support device 130 for use with one or more catheter tubes.

Here, the gripping members 53, 54 are connected to either side of the base member 52. The gripping members 53,54 may be integral with or attached to the base member 52 via a flexible hinge or seam region.

The gripping members 53, 54 extend from protrusions on either side of the base member 52. As with the first and second embodiment, the gripping members 53, 54 are preferably of the general form of flexible straps or covers. However, the attachment regions 58, 59 are attachable to a patent's body.

In use, each gripping member 53, 54 is extended over a catheter placed on the base member 52 and then attached to the patent's body at the attachment regions 58, 59, as illustrated in FIG. 13.

As discussed previously, the gripping members 53, 54 are provided with intermediate region 56, 57 between the connection to the base member 52 and the attachment region 58, 59.

The intermediate regions 56, 57 may each be provided with a preformed curvature to substantially correspond with the outside circumference of a catheter tube, thereby aiding the location and gripping of the catheter tubes. As described above in relation to the first embodiment, the underside of the intermediate regions 56, 57 may also, or alternatively, be provided with additional gripping means to enhance grip. In the currently preferred embodiment, the undersides of the intermediate regions 56, 57 are substantially free of adhesive, thereby enabling the catheter tubes to be gripped without leaving any sticky residue on the catheters when removed.

The base member 52 may be provided with a cover part as described previously with reference to FIG. 11. Further, the base member 52 may be uniformly covered with adhesive or provided with one or more adhesive regions, as described with reference to FIG. 11 for attachment to the patient's body.

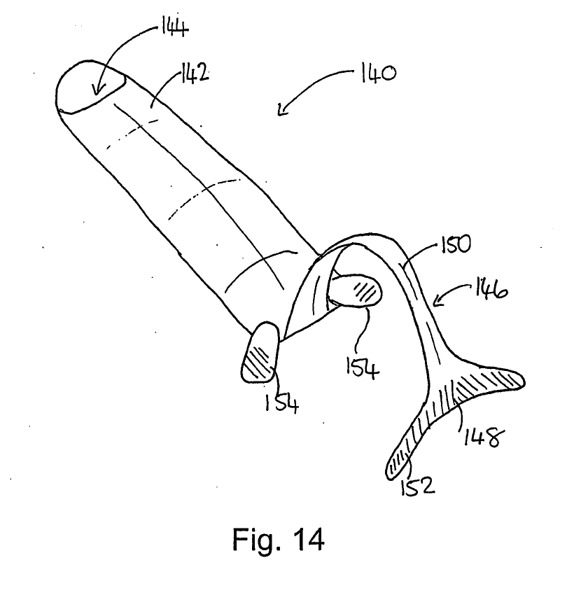

FIG. 14 illustrates a fourth embodiment of a support dressing or support device 140 for use with one or more catheter tubes.

In this embodiment the base member comprises a pocket 142. Pocket 142 has at least one open end 144 into which a catheter can be inserted.

Preferably a gripping member 146 is provided. The gripping member 146 is preferably of the general form of flexible straps or covers

The gripping member 146 comprises an attachment region 148, of which the undersides may be attachable to the base member 142—for example attaching to the top of the pocket. Between the attachment to the pocket 146 and the attachment regions 148, the gripping member 146 incorporates an intermediate region 150 that is adapted to wrap over the pocket in use.

The intermediate regions 150 may be provided with a preformed curvature to substantially correspond with the outside circumference of a catheter tube, thereby aiding the location and gripping of the catheter tubes. As described above in relation to the first embodiment, the underside of the intermediate regions 150 may also, or alternatively, be provided with additional gripping means to enhance grip.

Preferably, the attachment region 148 includes projections 152 enabling the gripping member 146 to be attached to a patient's body.

As discussed with reference to earlier embodiments, the base member (pocket) 142 may be uniformly covered with adhesive or be provided with one or more adhesive regions. As illustrated in FIG. 14 the adhesive regions may be provided upon projections 154 which are integral to the base member 142 or attached to the base member, for example using stitching or an adhesive.

The base member may also be provided with a cover part as described with reference to FIGS. 11a and 11b.

The material used to make the device (and indeed any of the devices described herein) may be impregnated or treated with an anti-infection or antibacterial agent to reduce the likelihood of infection to the patient. Possible anti-infection or antibacterial agents are described below. Whilst the entire device may be impregnated or treated with an anti-infection or antibacterial agent, it will be appreciated that it may only be necessary to impregnate or treat the cover part in this manner, since bacteria or other causes of infection will only enter the human body through the catheter exit sites in the skin, or through the Luer locks at the end of the catheter lines. Thus, covering the catheter exit sites with a cover part impregnated or treated with an anti-infection or antibacterial agent helps to prevent infection from getting into the human body and the bloodstream.

General Features and Options Applicable to all Embodiments

The Attachment Regions of the Gripping Members

As discussed above, the attachment region(s) of each gripping member may be coated with adhesive. Alternatively, or in addition, the base member may be coated with adhesive in a region or regions to correspond with the attachment region(s). The adhesive may be such as to allow the gripping member(s) to be attached to and then detached from the base member, thereby enabling the catheter tube(s) to be easily removed from the patient's body.

Moreover, the adhesive may be such as to allow the gripping member(s) to be reversibly attached to, detached from, and then re-attached to the base member. Such an adhesive not only enables the catheter tube(s) to be easily removed from the patient's body, but also readily enables the tubes to be adjusted or repositioned on the patient, thereby enabling a medical professional to fit the tubes in an optimum manner for operation and patient comfort.

As an alternative to using such adhesive, the inner surface(s) (e.g. 20) of the attachment region(s) of the gripping member(s) may incorporate hook-and-loop fastener material (e.g. Velcro (RTM)), and complementary hook-and-loop fastener material may be applied to the base member (e.g. in region 22) to reversibly engage with the hook-and-loop fastener material provided on the respective gripping member. This enables the device to be repeatedly unfastened and refastened around a catheter, and for the catheter to be easily removed, adjusted or repositioned as required.

The attachment region(s) need not be at the far end of each gripping member. Indeed, a gripping member may extend past the attachment region, away from the catheter tube in use, in order to provide a tab or handle by which to pull open the device and thereby release the catheter. Such a tab or handle may extend outwardly beyond the base member.

Also, the region(s)of the base member onto which the attachment region(s) attach, need not be at the edge of the base member. Thus, any tab or handle regions of the gripping member(s) may be free to be pulled, but may nevertheless be within the footprint area of the base member.

The Base Member

The underside of the base member (e.g. 24 in FIG. 1) is preferably covered with standard medical adhesive. The base member may be affixed to a haemodialysis patient for two to three days at a time, and taken off and changed when appropriate. However, if a reversible adhesive or hook-and-loop fastener material is used on the attachment regions of the gripping members, then it may be possible to leave the base member attached to the patient for a week or more.

With the dual catheter device, the size of the base member is anticipated to be of the order of 7 cm by 7 cm. With the single catheter device 10, the base member is anticipated to be of the order of 4.5 cm wide by 7 cm long. However, it should be emphasised that these dimensions are merely approximate examples, and the devices could be made in many other shapes and sizes as appropriate for the intended medical use.

Additional gripping means may be provided on the upper surface of the base member, made of similar material to the gripping means described above, and positioned so as to contact a catheter tube in use and thereby further enhance the grip between the device and the catheter.

Manufacturing Materials

The support dressings or support devices may be made from any suitable one or more of the following materials:
gauze, paper, reinforced paper, cardboard, woven fabric, non-woven fabric, plastic, terry cloth.

Other suitable materials may be readily apparent to those skilled in the art of medical devices and dressings.

The material may be impregnated or treated with an anti-infection or antibacterial agent to reduce the likelihood of infection to the patient. The anti-infection or antibacterial agent may be a silver-based agent such as silver sulphadiazine, although other suitable silver-based agents, and indeed non-silver-based agents, will be familiar to those skilled in the art. Alternatively, or in addition, the material may be impregnated or treated with a waterproofing agent, to protect the device from becoming dysfunctional should it become wet.

Summary of Benefits

The catheter support dressings or support devices described herein provide inter alia the following benefits when compared to known catheter support dressings or devices:
The catheter line is easier to install.
The catheter line can be held more securely.
Less (or no) damage is caused to the catheter line.
Less (or no) adhesive residue remains on the catheter line when removed.
The device may be pliable and easily manipulated.
The device is more comfortable for the patient to wear, and is more reassuring to him/her.
The catheter line can be held in a neater, less unsightly manner.
There is less movement of the catheter line when secured, thereby leading to a reduced likelihood of infection for the patient
The device may be used reversibly, enabling the catheter line to be removed or adjusted, and subsequently secured again.
Through using such a device, because a catheter does not become coated with adhesive residue or otherwise damaged, the catheter may have a longer useful life, thereby requiring less frequent replacement and consequently saving money.
The device can also cover the catheter exit site as well as securing the catheter against the patient's body.
The device provides the first dedicated dialysis line dressing.

1-21. (canceled)

22. A device for securing a catheter tube against a patient's body, said device comprising a base member for attaching to the patient's body in use, and a first gripping member, the first gripping member comprising:
a first region that is attached to, or attachable to, or integral with, the base member;
an attachment region that is attachable to the base member; and
an intermediate region between the first region and the attachment region, the intermediate region being adapted to wrap over a catheter tube;
the device further comprising a cover part for covering the site(s) at which one or more catheter tubes emerge from the human body, and being arranged such that, in use, the first region of the first gripping member is attached to the base member, the intermediate region is wrapped over a catheter, and the attachment region is attached to the base member, thereby securing the catheter tube between the base member and the first gripping member.

23. A device as claimed in claim 22 further adapted to secure two catheter tubes against a patient's body, and comprising a base member for attaching to the patient's body in use, a second gripping member, the second gripping member comprising:
a first region that is attached to, or attachable to, or integral with, the base member;
an attachment region that is attachable to the base member; and
an intermediate region between the first region and the attachment region, the intermediate region being adapted to wrap over a catheter tube;
the device being arranged such that, in use, the first region of the second gripping member is attached to the base member, the intermediate region of the first and second gripping member is wrapped over a respective catheter tube, and the attachment region of the second gripping member is attached to the base member, thereby securing the two catheter tubes between the base member and the respective gripping members.

24. A device as claimed in claim 23, wherein the gripping members are attached in substantially the centre of the base member.

25. A device as claimed in claim 22, wherein the attachment region or regions is/are substantially flat.

26. A device as claimed in claim 22, wherein the attachment region or regions is/are coated with adhesive.

27. A device as claimed in claim 22, wherein the base member is coated with adhesive in a region or regions to correspond with the attachment region or regions.

28. A device as claimed in claim 26, wherein the adhesive is adapted to allow the gripping member or members to be attached to and then detached from the base member.

29. A device as claimed in claim 28, wherein the adhesive is adapted to allow the gripping member or members to be attached to, detached from, and then re-attached to the base member.

30. A device as claimed in claim 22, wherein the attachment region or regions incorporate hook-and-loop fastener material, and the base member incorporates complementary hook-and-loop fastener material.

31. A device as claimed in claim 22, wherein the or each intermediate region incorporates a preformed curvature to substantially correspond with the outside circumference of a catheter tube.

32. A device as claimed in claim 22, wherein the or each intermediate region incorporates gripping means on its underside, for enhancing the grip between the gripping member and a catheter tube in use.

33. A device as claimed in claim 22, wherein the base member incorporates gripping means on its upper surface, for enhancing the grip between the device and a catheter tube in use.

34. A device as claimed in claim 32, wherein the gripping means are selected from a group comprising: padding, foam, rubber, adhesive.

35. A device as claimed in claim 22, wherein the or each gripping member extends outwardly beyond the base member.

36. A device as claimed in claim 1, impregnated or treated with an anti-infection or antibacterial agent.

37. A device as claimed in claim 22, impregnated or treated with a waterproofing agent.

38. A device as claimed in claim 22, made of a material selected from a group comprising: gauze, paper, reinforced paper, cardboard, woven fabric, non-woven fabric, plastic, terry cloth.

39. A device as claimed in claim 22, wherein the underside of the base member incorporates adhesive for attaching the device to the patient's body in use.

40. A device configured to secure a catheter tube against a patient's body and also cover the site at which the catheter tube emerges from the patient's body.

41. A device for securing a catheter tube against a patient's body, said device comprising a base member for attaching to the patient's body in use, and a first gripping member, the first gripping member comprising:
a first region that is attached to, or attachable to, or integral with, the base member;
an attachment region that is attachable to the base member; and
an intermediate region between the first region and the attachment region, the intermediate region being adapted to wrap over a catheter tube;
the device being arranged such that, in use, the first region of the gripping member is attached to the base member, the intermediate region is wrapped over a catheter, and the attachment region is attached to the base member, such that the device forms a pocket into which a catheter can be inserted.

42. A method of securing a catheter tube against a patient's body, said method comprising:
obtaining a device as claimed in any preceding claim;
attaching a first region of a gripping member to the base member or receiving the device with the first region of a gripping member pre-attached to the base member;
attaching the base member to the patient;
wrapping the gripping member over the catheter tube; and
attaching the attachment region of the gripping member to the base member, thereby securing the catheter tube between the base member and the gripping member.
